(19) United States
(12) Patent Application Publication
(10) Pub. No.: US 2026/0308195 A1
(43) Pub. Date: Oct. 8, 2026

(54) METHOD FOR PURIFYING CHRYSOMYCIN A FROM FERMENTATION PRODUCTS OF STREPTOMYCES

(71) Applicant: Zhejiang University of Technology, Hangzhou (CN)

(72) Inventors: Huawei Zhang, Hangzhou (CN); Zhehui Cai, Hangzhou (CN); Yuan Kai, Hangzhou (CN); Hong Wang, Hangzhou (CN)

(21) Appl. No.: 19/273,160

(22) Filed: Jul. 18, 2025

(30) Foreign Application Priority Data
Mar. 27, 2025 (CN) 202510370560.4

(51) Int. Cl. C07H 7/06; C07H 1/08; C12N 1/20; C12P 19/02; C12R 1/465

(52) U.S. Cl. CPC C07H 7/06; C07H 1/08; C12N 1/205; C12P 19/02; C12R 2001/465

(57) ABSTRACT

An efficient method for purifying chrysomycin A from fermentation products of Streptomyces is provided. The method only requires drying mycelia, followed by ultrasonic extraction, concentration and crystallization, redissolution in an organic solvent, and recrystallization. The method is simple to operate, has low equipment requirements, involves low consumable costs, and allows for convenient recovery of the organic solvent. The method overcomes drawbacks of existing methods of complicated steps of liquid phase preparative chromatography separation and purification, high equipment requirements, and longtime consumption. After a first crystallization and washing, a purity of the chrysomycin A in an obtained crude product of chrysomycin A is 87% to 92%. After recrystallization and a second washing of the crude product of chrysomycin A, the purity of the chrysomycin A reaches above 95%. An overall yield is over 47%, and a total time consumption is 90 hours to 144 hours.

### CROSS-REFERENCE TO RELATED APPLICATION

This application claims priority to Chinese Patent Application No. 202510370560.4, filed on Mar. 27, 2025, which is herein incorporated by reference in its entirety.

### TECHNICAL FIELD

The disclosure relates to the technical field of chemical separation and purification, and more particularly to a method for purifying chrysomycin A from fermentation products of Streptomyces.

### BACKGROUND

Chrysomycin A is a fermentation product derived from Streptomyces, which has significant anti-tumor and anti-gram-positive bacteria activities and has great potential for new medicine development. However, in fermentation products of Streptomyces producing chrysomycin A, there are usually two by-products, chrysomycin B and chrysomycin C. Structural formulas of chrysomycin A, chrysomycin B and chrysomycin C are as follows:

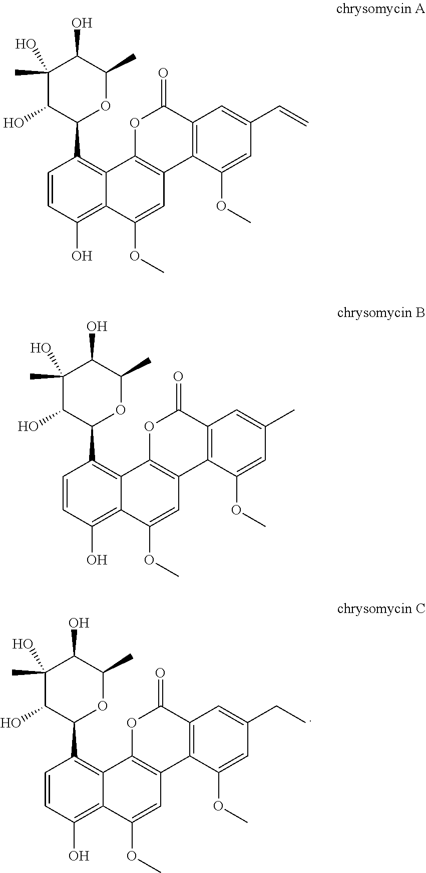

Because the structure of chrysomycin A is very similar to those of chrysomycin B and chrysomycin C, it is extremely difficult to prepare high-purity chrysomycin A by separation and purification methods in the related art. Currently, large-scale separation and purification of high-purity chrysomycin A at home and abroad mainly depends on preparative chromatography separation. The preparative chromatography separation requires complicated pretreatment processes such as concentrating an extract to dry and redissolving the extract for extraction and decolorization. The preparative chromatography separation has disadvantages of high technical equipment requirements, high cost of consumables, complicated operation and longtime consumption.

### SUMMARY

The disclosure aims to provide an efficient method for purifying chrysomycin A from fermentation products of Streptomyces, which avoids use of liquid chromatography and reagents in separation and purification process of chrysomycin A, solves problems of cumbersome steps, high cost and longtime consumption of methods in the art, and develops a method for separation and purification of chrysomycin A totally different from the related art.

Technical solutions provided by the disclosure are as follows.

The efficient method for purifying the chrysomycin A from the fermentation products of Streptomyces provided by the disclosure includes the following steps:
(1) filtering the fermentation products of Streptomyces containing the chrysomycin A to collect wet mycelium; vacuum drying the wet mycelium to obtain first mycelium; and crushing the first mycelium (screening with an 80-mesh sieve) to obtain dried mycelium;
(2) performing ultrasonic extraction on the dried mycelium with a first organic solvent to obtain a first mixture; and filtering the first mixture to obtain an extract;
(3) concentrating the extract to a supersaturated state to obtain a concentrated extract; standing the concentrated extract at room temperature for crystallization to obtain a crystallized extract; filtering the crystallized extract to obtain first crystal; and washing the first crystal with a first solvent to remove impurities, to thereby obtain a crude product of chrysomycin A; and
(4) redissolving the crude product of chrysomycin A with a second organic solvent to obtain a second mixture; filtering the second mixture to remove insoluble impurities to thereby obtain a filtered second mixture; concentrating the filtered second mixture to the supersaturated state to obtain a concentrated second mixture; standing the concentrated mixture at the room temperature for recrystallization to obtain a recrystallized second mixture; filtering the recrystallized second mixture to obtain second crystal; and washing the second crystal with a second solvent to remove impurities, to thereby obtain a pure product of chrysomycin A.

In an embodiment, in step (1), the fermentation products of Streptomyces containing the chrysomycin A are obtained by fermentation of Streptomyces sp. 891-B6 with a deposit number of China General Microbiological Culture Collection Center (CGMCC) No. 21775.

In an embodiment, in the step (1), the fermentation products of Streptomyces are prepared by the following steps: inoculating Streptomyces sp. 891-B6 with the deposit number of CGMCC No. 21775 onto an international streptomyces projects (ISP) 2 agar medium for activation for 6 days (d) to 7 d to thereby obtain activated Streptomyces; then inoculating the activated Streptomyces onto an ISP2 liquid medium for seed culture at 28 Celsius degrees (° C.) with a shaking speed at 200 revolutions per minute (rpm) for 48 hours (h) to 72 h to obtain a seed solution; and subsequently, inoculating the seed solution onto a fermentation medium at a volume concentration of 5% for fermentation at 28° C. with a shaking speed at 200 rpm for 168 h to 192 h, to thereby obtain a fermentation broth as the fermentation products of Streptomyces;
in which, the ISP2 agar medium is composed of: glucose with a concentration of 4.0 grams per liter (g/L), yeast extract with a concentration of 4.0 g/L, malt extract with a concentration of 10.0 g/L and agar powder with a concentration of 20.0 g/L; a potential of hydrogen (pH) value of the ISP2 agar medium is 7.0; and the ISP2 agar medium is sterilized at 121° C. for 20 minutes (min);
in which, the ISP2 liquid medium is composed of: the glucose with the concentration of 4.0 g/L, the yeast extract with the concentration of 4.0 g/L, and the malt extract with the concentration of 10.0 g/L; a pH value of the ISP2 liquid medium is 7.0; and the ISP2 liquid medium is sterilized at 121° C. for 20 min; and
in which, the fermentation medium includes: starch with a concentration of 5.0 g/L, the glucose with a concentration of 20.0 g/L, soybean powder with a concentration of 10.0 g/L, and calcium carbonate with a concentration of 2.0 g/L; a pH value of the fermentation medium is 7.0; and the fermentation medium is sterilized at 121° C. for 20 min.

In an embodiment, the wet mycelium is vacuum dried at a temperature in a range of 50° C. to 70° C. for 48 h to 72 h. Specifically, the wet mycelium is vacuum dried at 60° C. for 48 h.

In an embodiment, in step (2), the first organic solvent is one or more selected from the group consisting of methanol, ethanol, propanol, butanol, ethyl acetate, acetone and acetonitrile. A volume dosage of the first organic solvent is in a range of 0.1 liters per gram (L/g) to 0.5 L/g based on a weight of the dried mycelium.

In an embodiment, in step (4), the second organic solvent is one or more selected from the group consisting of methanol, ethanol, propanol, butanol, ethyl acetate, acetone and acetonitrile. A volume dosage of the second organic solvent is in a range of 1 L/g to 5 L/g based on a weight of the crude product of chrysomycin A.

In an embodiment, in the step (2), the ultrasonic extraction is performed at a power in a range of 100 watts (W) to 200 W for 10 min to 30 min. A process of the ultrasonic extraction is repeated for 1 time to 3 times.

In an embodiment, methods for filtering in the step (1) through the step (4) each are one of plate-frame pressure filtering, vacuum suction filtering and centrifugal filtering.

In an embodiment, in the step (3), the extract is concentrated until a concentration of the chrysomycin A is in a range of 5 g/L to 10 g/L. Purity and mass recovery rate of the chrysomycin A are compromised when the concentration of chrysomycin A is lower than 5 g/L or higher than 10 g/L.

In an embodiment, the first solvent in the step (3) and the second solvent in the step (4) each are one or more selected from the group consisting of water, methanol, ethanol, propanol, butanol, ethyl acetate, acetone, acetonitrile, n-hexane, petroleum ether, dichloromethane and chloroform.

In an embodiment, methods for the washing in the step (3) and the step (4) each are leaching or slurry washing.

In an embodiment, in the step (4), the filtered second mixture is concentrated until the concentration of the chrysomycin A is in a range of 4 g/L to 12 g/L.

In an embodiment, when the first organic solvent in the step (2) is ethanol the first solvent in the step (3) is pure water, the second organic solvent in the step (4) is ethyl acetate, and the second solvent in the step (4) is ethyl acetate. When the first organic solvent in the step (2) is methanol, the first solvent in the step (3) is ethanol, the second organic solvent in the step (4) is methanol, and the second solvent in the step (4) is acetonitrile. When the first organic solvent in the step (2) is ethanol, the first solvent in the step (3) is ethanol, the second organic solvent in the step (4) is ethanol, and the second solvent in the step (4) is ethanol.

“Concentrating” mentioned in the disclosure refers to a method of improving the concentration of chrysomycin A by solvent evaporation, including technical means such as vacuum evaporation and heating evaporation.

Compared with the related art, the disclosure has the following beneficial effects. The method for purifying the chrysomycin A from the fermentation products of Streptomyces provided by the disclosure only requires drying the fermentation products, followed by ultrasonic extraction, concentration and crystallization, redissolution in an organic solvent, and recrystallization. The method is simple to operate, has low equipment requirements, involves low consumable costs, and allows for convenient recovery of the organic solvent. The method overcomes drawbacks of methods in the art of complicated steps of liquid phase preparative chromatography separation and purification, high equipment requirements, and longtime consumption. After a first crystallization and washing, the purity of the chrysomycin A in the crude product of chrysomycin A is 87% to 92%. After recrystallization and a second washing of the crude product of chrysomycin A, the purity of the chrysomycin A reaches above 95%. An overall yield is over 47%. Therefore, the method is suitable for large-scale preparation of chrysomycin A with low cost and high efficiency. A total time consumption is 90 h to 144 h.

### BRIEF DESCRIPTION OF DRAWINGS

FIG. 1 illustrates a flowchart of purification of chrysomycin A.

FIG. 2 illustrates a liquid chromatogram of a crude product of chrysomycin A prepared by an embodiment 2 of the disclosure.

FIG. 3 illustrates a liquid chromatogram of a pure product of chrysomycin A prepared by the embodiment 2 of the disclosure.

FIG. 4 illustrates a liquid chromatogram of a crude product of chrysomycin A prepared by an embodiment 3 of the disclosure.

FIG. 5 illustrates a liquid chromatogram of a pure product of chrysomycin A prepared by the embodiment 3 of the disclosure.

FIG. 6 illustrates a liquid chromatogram of a crude product of chrysomycin A prepared by an embodiment 4 of the disclosure.

FIG. 7 illustrates a liquid chromatogram of a pure product of chrysomycin A prepared by the embodiment 4 of the disclosure.

FIG. 8 illustrates a liquid chromatogram of a crude product of chrysomycin A prepared by a comparative embodiment 1.

FIG. 9 illustrates a liquid chromatogram of a crude product of chrysomycin A prepared by a comparative embodiment 2.

FIG. 10 illustrates a liquid chromatogram of a pure product of chrysomycin A prepared by the comparative embodiment 2 of the disclosure.

FIG. 11 illustrates a liquid chromatogram of an extract prepared by a comparative embodiment 4.

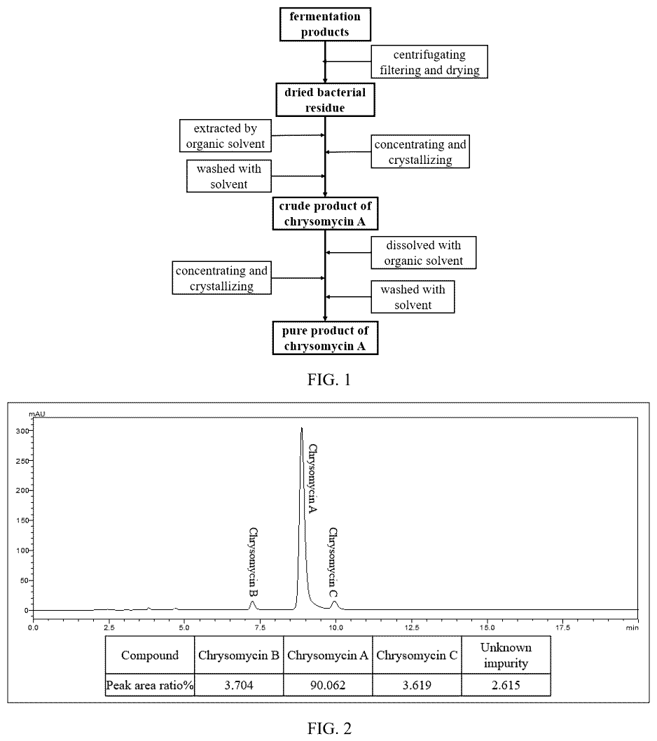

### DETAILED DESCRIPTION OF EMBODIMENTS

The disclosure will be further described as follows with reference to embodiments, but a scope of protection of the disclosure is not limited thereto.

A room temperature in the disclosure refers to 25° C. to 30° C.

Content, purity and mass recovery rate of chrysomycin A are detected by high performance liquid chromatography, specifically as follows.

A chromatograph is Shimadzu® LC-20. A chromatographic column is Luna-C18. A particle size is 5 micrometers (μm). The chromatographic column has a diameter of 4.6 millimeters (mm) and a height of 250 mm. An injection volume is 5 microliters (μL). A mobile phase consists of acetonitrile and water in a volume ratio of 50:50. A detection wavelength is 254 nanometers (nm). A retention time is 20 min. A pump flow rate is 1 milliliter per minute (mL/min). An order of peak emergence is chrysomycin B, followed by the chrysomycin A, and then chrysomycin C.

According to a standard curve of chrysomycin A made by a concentration and peak area of the chrysomycin A, the content, purity and mass recovery rate of chrysomycin A are calculated.

The purity of chrysomycin A is characterized by a peak area ratio provided by liquid chromatography.

### Embodiment 1: Preparation of a Fermentation Broth of Streptomyces

Streptomyces sp. 891-B6 with a deposit number of CGMCC No. 21775 (it has been disclosed in a Chinese patent with publication No. CN113278545A) is inoculated onto an ISP2 agar medium for activation for 6 d to 7 d to thereby obtain activated Streptomyces. The activated Streptomyces is inoculated onto an ISP2 liquid medium for seed culture at 28° C. with a shaking speed at 200 rpm for 48 h to 72 h to obtain a seed solution. The seed solution is inoculated onto a fermentation medium at a volume concentration of 5% for fermentation at 28° C. with a shaking speed at 200 rpm for 168 h to 192 h, to thereby obtain the fermentation broth as the fermentation products of Streptomyces.

The ISP2 agar medium is composed of: glucose with a concentration of 4.0 g/L, yeast extract with a concentration of 4.0 g/L, malt extract with a concentration of 10.0 g/L and agar powder with a concentration of 20.0 g/L. A pH value of the ISP2 agar medium is 7.0. The ISP2 agar medium is sterilized at 121° C. for 20 min.

The ISP2 liquid medium is composed of: the glucose with the concentration of 4.0 g/L, the yeast extract with the concentration of 4.0 g/L, and the malt extract with the concentration of 10.0 g/L. A pH value of the ISP2 liquid medium is 7.0. The ISP2 liquid medium is sterilized at 121° C. for 20 min.

The fermentation medium is composed of: starch with a concentration of 5.0 g/L, the glucose with a concentration of 20.0 g/L, soybean powder with a concentration of 10.0 g/L, and calcium carbonate with a concentration of 2.0 g/L. A pH value of the fermentation medium is 7.0. The fermentation medium is sterilized at 121° C. for 20 min.

### Embodiment 2: Separation and Purification of Chrysomycin A

(1) 37 liters (L) of the fermentation broth prepared in embodiment 1 is centrifuged at 25° C. with a shaking speed of 3500 rpm for 20 min. Supernatant is removed to obtain wet mycelium. The wet mycelium is vacuum dried at 60° C. for 48 h to obtain first mycelium. The first mycelium is crushed and then screened with an 80-mesh sieve to obtain dried mycelium with a weight of 505 grams (g).

(2) 1 g of the dried mycelium is added into 300 milliliters (mL) of ethanol to obtain a first mixture. The first mixture is performed with ultrasonic extraction at a power of 150 W for 25 min. Subsequently, the first mixture is filtered to obtain a filter cake. The filter cake is repeatedly extracted with a same amount of ethanol for 3 times and filtered to obtain 850 mL ethanol extract containing 0.11 g chrysomycin A.

(3) the 850 mL ethanol extract prepared in step (2) is vacuum evaporated and concentrated to 20 mL to obtain a concentrated extract. In the concentrated extract, a concentration of the chrysomycin A is about 5.5 g/L. The concentrated extract is stood at a room temperature for crystallization to obtain a crystallized extract. The crystallized extract is filtered to obtain first wet crystal. The first wet crystal is washed with pure water, to thereby obtain a crude product of chrysomycin A with a weight of 77.6 milligrams (mg). 1 mg of the crude product of chrysomycin A is redissolved with the ethanol and then detected by liquid chromatography. Results show that a purity of the chrysomycin A is 90.1%, and a mass recovery rate is 70.5%, as illustrated in FIG. 2.

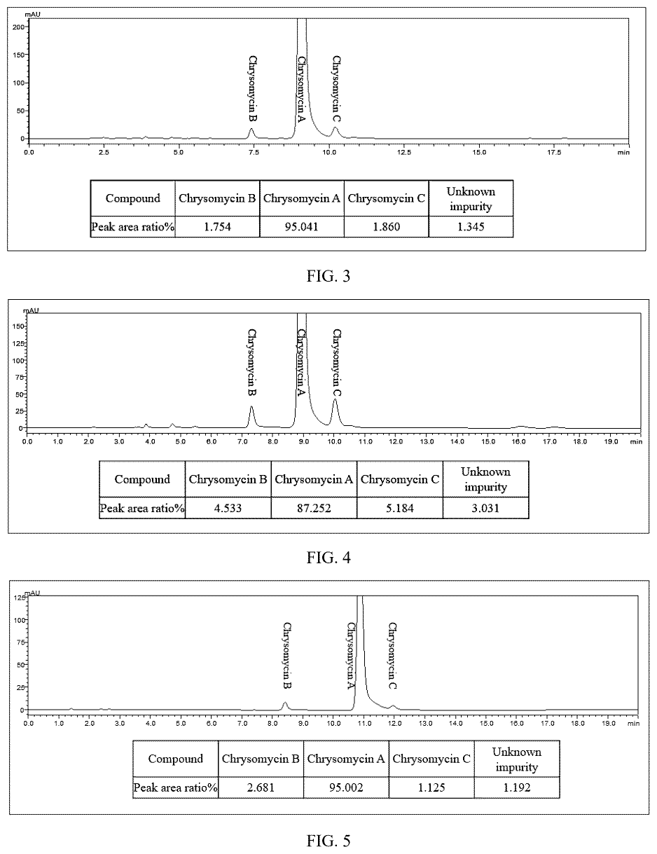

(4) 76.6 mg of the crude product of chrysomycin A is dissolved with 300 mL of ethyl acetate to obtain a second mixture (i.e., ethyl acetate solution). The second mixture is filtered to remove insoluble impurities to thereby obtain a filtered second mixture. The filtered second mixture is concentrated to 15 mL (the concentration of the chrysomycin A is 5.1 g/L) to obtain a concentrated second mixture. The concentrated second mixture is stood at the room temperature for recrystallization to obtain a recrystallized second mixture. The recrystallized second mixture is filtered to obtain second wet crystal. The second wet crystal is washed with a small amount of ethyl acetate to obtain a pure product of chrysomycin A with a weight of 57.7 mg. A total time consumption is 92 h. 1 mg of the pure product of chrysomycin A is redissolved with the ethanol and then detected by liquid chromatography. Results show that a purity of the chrysomycin A is 95.0%, and a total mass recovery rate is 52.5%, as illustrated in FIG. 3.

### Embodiment 3: Separation and Purification of Chrysomycin A

(1), 37 L of the fermentation broth prepared in embodiment 1 is centrifuged at 25° C. with a shaking speed of 3500 rpm for 20 min. Supernatant is removed to obtain wet mycelium. The wet mycelium is vacuum dried at 60° C. for 48 h to obtain first mycelium. The first mycelium is crushed and then screened with an 80-mesh sieve to obtain dried mycelium with a weight of 505 g.

(2), 10 g of the dried mycelium is added into 3 L of methanol to obtain a first mixture. The first mixture is performed with ultrasonic extraction at a power of 150 W for 25 min. Subsequently, the first mixture is filtered to obtain a filter cake. The filter cake is repeatedly extracted with a same amount of methanol for 3 times and filtered to obtain 8.8 L methanol extract containing 1.12 g chrysomycin A.

(3), the 8.8 L methanol extract prepared in step (2) is concentrated to 120 mL to obtain a concentrated extract. In the concentrated extract, a concentration of the chrysomycin A is 9.3 g/L. The concentrated extract is stood at a room temperature for crystallization to obtain a crystallized extract. The crystallized extract is filtered to obtain first wet crystal. The first wet crystal is washed with a small amount of ethanol, to thereby obtain a crude product of chrysomycin A with a weight of 0.85 g. 1 mg of the crude product of chrysomycin A is redissolved with the ethanol and then detected by liquid chromatography. Results show that a purity of the chrysomycin A is 87.3%, and a mass recovery rate is 75.9%, as illustrated in FIG. 4.

(4), 0.85 g of the crude product of chrysomycin A is dissolved with 1 L of methanol to obtain a second mixture (i.e., methanol solution). The second mixture is filtered to remove insoluble impurities to thereby obtain a filtered second mixture. The filtered second mixture is concentrated to 75 mL (the concentration of the chrysomycin A is 11.3 g/L) to obtain a concentrated second mixture. The concentrated second mixture is stood at the room temperature for recrystallization to obtain a recrystallized second mixture. The recrystallized second mixture is filtered to obtain second wet crystal. The second wet crystal is washed with a small amount of acetonitrile to obtain a pure product of chrysomycin A with a weight of 0.68 g. A total time consumption is 107 h. 1 mg of the pure product of chrysomycin A is redissolved with the ethanol and then detected by liquid chromatography. Results show that a purity of the chrysomycin A is 95.0%, and a total mass recovery rate is 60.7%, as illustrated in FIG. 5.

### Embodiment 4: Separation and Purification of Chrysomycin A

(1), 37 L of the fermentation broth prepared in embodiment 1 is centrifuged at 25° C. with a shaking speed of 3500 rpm for 20 min. Supernatant is removed to obtain wet mycelium. The wet mycelium is vacuum dried at 60° C. for 48 h to obtain first mycelium. The first mycelium is crushed and then screened with an 80-mesh sieve to obtain dried mycelium with a weight of 505 g.

(2), 100 g of the dried mycelium is added into 30 L of ethanol to obtain a first mixture. The first mixture is performed with ultrasonic extraction at a power of 150 W for 25 min. Subsequently, the first mixture is filtered to obtain a filter cake. The filter cake is repeatedly extracted with a same amount of ethanol for 3 times and filtered to obtain 89 L ethanol extract containing 11.03 g chrysomycin A.

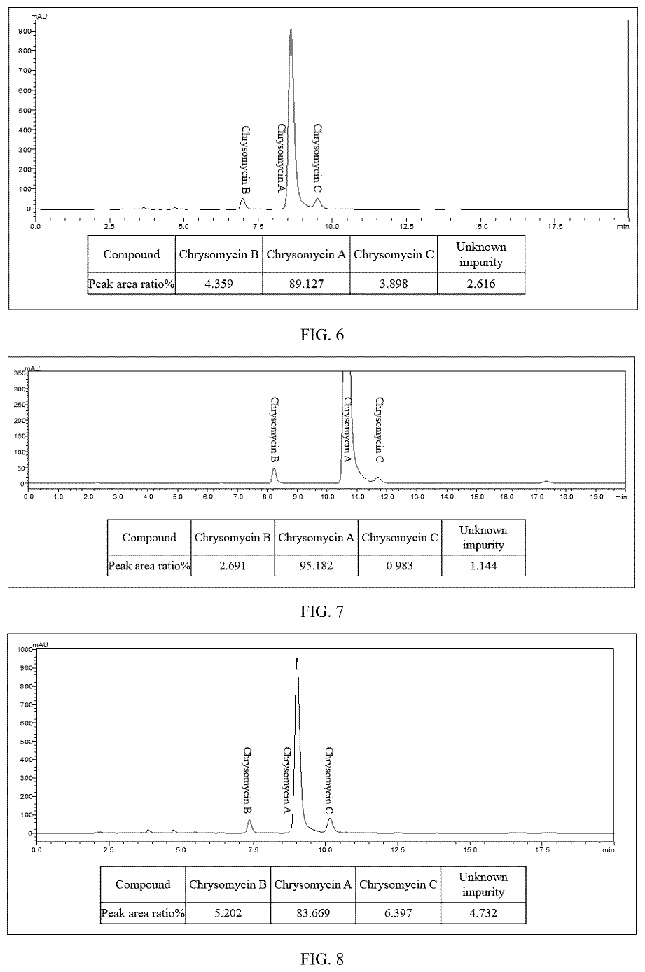

(3), the 89 L ethanol extract prepared in step (2) is concentrated to 1.5 L to obtain a concentrated extract. In the concentrated extract, a concentration of the chrysomycin A is 7.3 g/L. The concentrated extract is stood at a room temperature for crystallization to obtain a crystallized extract. The crystallized extract is filtered to obtain first wet crystal. The first wet crystal is washed with a small amount of ethanol, to thereby obtain a crude product of chrysomycin A with a weight of 8.05 g. 1 mg of the crude product of chrysomycin A is redissolved with the ethanol and then detected by liquid chromatography. Results show that a purity of the chrysomycin A is 89.1%, and a mass recovery rate is 73.0%, as illustrated in FIG. 6.

(4), 8.05 g of the crude product of chrysomycin A is dissolved with 10 L of ethanol to obtain a second mixture (i.e., ethanol solution). The second mixture is concentrated to 1.2 L (the concentration of the chrysomycin A is 5.9 g/L) to obtain a concentrated second mixture. The concentrated second mixture is stood at the room temperature for recrystallization to obtain a recrystallized second mixture. The recrystallized second mixture is filtered to obtain a second wet crystal. The second wet crystal is washed with a small amount of ethanol to obtain a pure product of chrysomycin A with a weight of 5.27 g. A total time consumption is 144 h. 1 mg of the pure product of chrysomycin A is redissolved with the ethanol and then detected by liquid chromatography. Results show that a purity of the chrysomycin A is 95.2%, a total mass recovery rate is 47.8%, as illustrated in FIG. 7.

### Comparative Embodiment 1: Effect of Concentration Degree on Separation and Purification of Chrysomycin A

(1), 37 L of the fermentation broth prepared in embodiment 1 is centrifuged at 25° C. with a shaking speed of 3500 rpm for 20 min. Supernatant is removed to obtain wet mycelium. The wet mycelium is vacuum dried at 60° C. for 48 h to obtain first mycelium. The first mycelium is crushed and then screened with an 80-mesh sieve to obtain dried mycelium with a weight of 505 g.

(2), 10 g of the dried mycelium is added into 3 L of methanol to obtain a first mixture. The first mixture is performed with ultrasonic extraction at a power of 150 W for 25 min. Subsequently, the first mixture is filtered to obtain a filter cake. The filter cake is repeatedly extracted with a same amount of methanol for 3 times and filtered to obtain 8.9 L methanol extract containing 1.13 g chrysomycin A.

(3), the 8.9 L methanol extract prepared in step (2) is concentrated to 80 mL to obtain a concentrated extract. In the concentrated extract, a concentration of the chrysomycin A is 14.1 g/L. When the concentration of the chrysomycin A is too high, a large amount of wet crystal is precipitated during concentrating the methanol extract. The concentrated extract is filtered to obtain the wet crystal. The wet crystal is washed with a small amount of ethanol, to thereby obtain a crude product of chrysomycin A with a weight of 0.62 g. 1 mg of the crude product of chrysomycin A is redissolved with the ethanol and then detected by liquid chromatography. Results show that a purity of the chrysomycin A is only 83.7%, and a mass recovery rate is only 54.9%, as illustrated in FIG. 8.

### Comparative Embodiment 2: Effect of Leaching on Separation and Purification of Chrysomycin A

(1), 37 L of the fermentation broth prepared in embodiment 1 is centrifuged at 25° C. with a shaking speed of 3500 rpm for 20 min. Supernatant is removed to obtain wet mycelium. The wet mycelium is vacuum dried at 60° C. for 48 h to obtain first mycelium. The first mycelium is crushed and then screened with an 80-mesh sieve to obtain dried mycelium with a weight of 505 g.

(2), 1 g of the dried mycelium is added into 300 mL of ethanol to obtain a first mixture. The first mixture is performed with ultrasonic extraction at a power of 150 W for 25 min. Subsequently, the first mixture is filtered to obtain a filter cake. The filter cake is repeatedly extracted with a same amount of ethanol for 3 times and filtered to obtain 850 mL ethanol extract containing 0.11 g chrysomycin A.

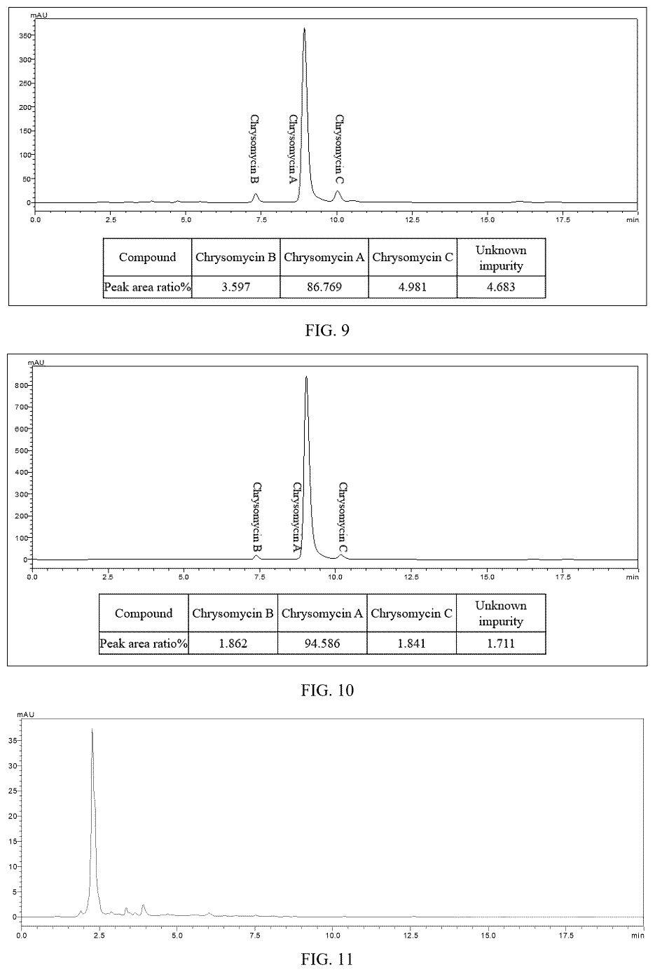

(3), the 850 mL ethanol extract prepared in step (2) is vacuum evaporated and concentrated to 20 mL to obtain a concentrated extract. In the concentrated extract, a concentration of the chrysomycin A is 5.5 g/L. The concentrated extract is stood at room temperature for crystallization to obtain a crystallized extract. First wet crystal is directly filtered out of the crystallized extract without washing, to thereby obtain a crude product of chrysomycin A with a weight of 56.5 mg, 1 mg of the crude product of chrysomycin A is redissolved with the ethanol and then detected by liquid chromatography. Results show that a purity of the chrysomycin A is 86.8%, and a mass recovery rate is 51.4%, as illustrated in FIG. 9. The purity of the chrysomycin A in this comparative embodiment is lower than that in the embodiment 2.

(4), 55.5 mg of the crude product of chrysomycin A is dissolved with 300 mL of ethanol to obtain a second mixture (i.e., ethanol solution). The second mixture is concentrated to 11 mL (the concentration of the chrysomycin A is 5.1 g/L) to obtain a concentrated second mixture. The concentrated second mixture is stood at temperature for recrystallization to obtain a recrystallized second mixture. Second wet crystal is directly filtered out of the recrystallized second mixture without washing, to thereby obtain a pure product of chrysomycin A with a weight of 38.4 mg, 1 mg of the pure product of chrysomycin A is redissolved with the ethanol and then detected by liquid chromatography. Results show that a purity of the chrysomycin A is 94.6%, and a total mass recovery rate is 34.9%. A total time consumption is 92 h. The results are illustrated in FIG. 10. The purity of the chrysomycin A does not reach 95.0%.

### Comparative Embodiment 3: Effect of Concentration Degree of Extract on Separation and Purification of Chrysomycin A

(1), 37 L of the fermentation broth prepared in embodiment 1 is centrifuged at 25° C. with a shaking speed of 3500 rpm for 20 min. Supernatant is removed to obtain wet mycelium. The wet mycelium is vacuum dried at 60° C. for 48 h to obtain first mycelium. The first mycelium is crushed and then screened with an 80-mesh sieve to obtain dried mycelium with a weight of 505 g.

(2), 10 g of the dried mycelium is added into 3 L of methanol to obtain a first mixture. The first mixture is performed with ultrasonic extraction at a power of 150 W for 25 min. Subsequently, the first mixture is filtered to obtain a filter cake. The filter cake is repeatedly extracted with a same amount of methanol for 3 times and filtered to obtain 8.9 L methanol extract containing 1.13 g chrysomycin A.

(3), the 8.9 L methanol extract prepared in step (2) is concentrated to 300 mL to obtain a concentrated extract. In the concentrated extract, a concentration of the chrysomycin A is 3.8 g/L. Because the concentration of the chrysomycin A is too low, no crystal is precipitated when the concentrated extract is stood at a room temperature for crystallization.

### Comparative Embodiment 4: Effect of Extract Agent on Separation and Purification of Chrysomycin A

(1), 37 L of the fermentation broth prepared in embodiment 1 is centrifuged at 25° C. with a shaking speed of 3500 rpm for 20 min. Supernatant is removed to obtain wet mycelium. The wet mycelium is vacuum dried at 60° C. for 48 h to obtain first mycelium. The first mycelium is crushed and then screened with an 80-mesh sieve to obtain dried mycelium with a weight of 505 g.

(2), 1 g of the dried mycelium is added into 300 mL of pure water to obtain a first mixture. The first mixture is performed with ultrasonic extraction at a power of 150 W for 25 min. Subsequently, the first mixture is filtered to obtain a filter cake. The filter cake is repeatedly extracted with a same amount of pure water for 3 times and filtered to obtain 890 mL extract. When detected by liquid chromatography, the extract does not contain chrysomycin A, as illustrated in FIG. 11.

What is claimed is:

1. A method for purifying chrysomycin A from fermentation products of Streptomyces, comprising the following steps:
(1) filtering the fermentation products of Streptomyces containing the chrysomycin A to collect wet mycelium; vacuum drying the wet mycelium to obtain first mycelium; and crushing the first mycelium to obtain dried mycelium;
(2) performing ultrasonic extraction on the dried mycelium with a first organic solvent to obtain a first mixture; and filtering the first mixture to obtain an extract;
(3) concentrating the extract to a supersaturated state to obtain a concentrated extract; standing the concentrated extract at a room temperature for crystallization to obtain a crystallized extract; filtering the crystallized extract to obtain first crystal; and washing the first crystal with a first solvent to remove impurities, to thereby obtain a crude product of chrysomycin A; and
(4) redissolving the crude product of chrysomycin A with a second organic solvent to obtain a second mixture; filtering the second mixture to remove insoluble impurities to thereby obtain a filtered second mixture; concentrating the filtered second mixture to the supersaturated state to obtain a concentrated second mixture; standing the concentrated second mixture at the room temperature for recrystallization to obtain a recrystallized second mixture; filtering the recrystallized second mixture to obtain second crystal; and washing the second crystal with a second solvent to remove impurities, to thereby obtain a pure product of chrysomycin A.

2. The method for purifying the chrysomycin A from the fermentation products of Streptomyces as claimed in claim 1, wherein, in step (1), the fermentation products of Streptomyces containing the chrysomycin A is obtained by fermentation of Streptomyces sp. 891-B6 with a deposit number of China General Microbiological Culture Collection Center (CGMCC) No. 21775.

3. The method for purifying the chrysomycin A from the fermentation products of Streptomyces as claimed in claim 1, wherein, in step (1), the fermentation products of Streptomyces are prepared by the following steps: inoculating Streptomyces sp. 891-B6 with a deposit number of CGMCC No. 21775 onto an international streptomyces projects (ISP) 2 agar medium for activation for 6 days (d) to 7 d to thereby obtain activated Streptomyces; then inoculating the activated Streptomyces onto an ISP2 liquid medium for seed culture at 28 Celsius degrees (° C.) with a shaking speed at 200 revolutions per minute (rpm) for 48 hours (h) to 72 h to obtain a seed solution; and subsequently, inoculating the seed solution onto a fermentation medium at a volume concentration of 5% for fermentation at 28° C. with a shaking speed at 200 rpm for 168 h to 192 h, to thereby obtain a fermentation broth as the fermentation products of Streptomyces;
wherein the ISP2 agar medium is composed of: glucose with a concentration of 4.0 grams per liter (g/L), yeast extract with a concentration of 4.0 g/L, malt extract with a concentration of 10.0 g/L and agar powder with a concentration of 20.0 g/L; a potential of hydrogen (pH) value of the ISP2 agar medium is 7.0; and the ISP2 agar medium is sterilized at 121° C. for 20 minutes (min);
wherein the ISP2 liquid medium is composed of: the glucose with the concentration of 4.0 g/L, the yeast extract with the concentration of 4.0 g/L, and the malt extract with the concentration of 10.0 g/L; a pH value of the ISP2 liquid medium is 7.0; and the ISP2 liquid medium is sterilized at 121° C. for 20 min; and
wherein the fermentation medium is composed of: starch with a concentration of 5.0 g/L, the glucose with a concentration of 20.0 g/L, soybean powder with a concentration of 10.0 g/L, and calcium carbonate with a concentration of 2.0 g/L; a pH value of the fermentation medium is 7.0; and the fermentation medium is sterilized at 121° C. for 20 min.

4. The method for purifying the chrysomycin A from the fermentation products of Streptomyces as claimed in claim 1, wherein, in step (2), the first organic solvent is one or more selected from the group consisting of methanol, ethanol, propanol, butanol, ethyl acetate, acetone and acetonitrile; and a volume dosage of the first organic solvent is in a range of 0.1 liters per gram (L/g) to 0.5 L/g based on a weight of the dried mycelium.

5. The method for purifying the chrysomycin A from the fermentation products of Streptomyces as claimed in claim 1, wherein, in step (4), the second organic solvent is one or more selected from the group consisting of methanol, ethanol, propanol, butanol, ethyl acetate, acetone and acetonitrile; and a volume dosage of the second organic solvent is in a range of 1 L/g to 5 L/g based on a weight of the crude product of chrysomycin A.

6. The method for purifying the chrysomycin A from the fermentation products of Streptomyces as claimed in claim 1, wherein, in step (2), the ultrasonic extraction is performed at a power in a range of 100 watts (W) to 200 W for 10 min to 30 min; and a process of the ultrasonic extraction is repeated for 1 time to 3 times.

7. The method for purifying the chrysomycin A from the fermentation products of Streptomyces as claimed in claim 1, wherein, in step (3), the extract is concentrated until a concentration of the chrysomycin A is in a range of 5 g/L to 10 g/L.

8. The method for purifying the chrysomycin A from the fermentation products of Streptomyces as claimed in claim 1, wherein, in step (4), the filtered second mixture is concentrated until a concentration of the chrysomycin A is in a range of 4 g/L to 12 g/L.

9. The method for purifying the chrysomycin A from the fermentation products of Streptomyces as claimed in claim 1, wherein the first solvent in step (3) and the second solvent in step (4) each are one or more selected from the group consisting of water, methanol, ethanol, propanol, butanol, ethyl acetate, acetone, acetonitrile, n-hexane, petroleum ether, dichloromethane and chloroform.

10. The method for purifying the chrysomycin A from the fermentation products of Streptomyces as claimed in claim 1, wherein methods for the washing in step (3) and step (4) each are leaching or slurry washing.
